(19) United States
(12) Patent Application Publication
(10) Pub. No.: US 2021/0115403 A1
(43) Pub. Date: Apr. 22, 2021

(54) METHOD FOR PREPARING AN IRRADIATED PLATELET LYSATE

(71) Applicant: MACO PHARMA, Mouvaux (FR)

(72) Inventors: Bruno Delorme, Marcq-en-Baroeul (FR); Sabrina Viau, Wasquehal (FR)

(21) Appl. No.: 16/992,175

(22) PCT Filed: Feb. 12, 2019
(86) PCT No.: PCT/EP2019/053471

§ 371 Date: Aug. 13, 2020

(30) Foreign Application Priority Data
Feb. 15, 2018 (FR) 1851306

(51) Int. Cl. C12N 5/078

(52) U.S. Cl. CPC C12N 5/0644; C12N 2529/00

(57) ABSTRACT

The invention relates to a method for preparing an irradiated platelet lysate, comprising the steps providing a platelet lysate to obtain a starting platelet lysate, the starting platelet lysate having platelet factors including growth factors and having plasma proteins including coagulation factors and proteins other than the coagulation factors. Irradiating the starting platelet lysate using UVC radiation with a wavelength of between 200 and 280 nm, in order to obtain a platelet lysate irradiated with UVC radiation. The irradiation being arranged to retain at least 75% of the total protein concentration of the starting platelet lysate, while reducing, by at least 20%, the concentration of at least one of the coagulation factors including fibrinogen, factor II, factor VII, factor IX, factor X and factor XI of the starting platelet lysate.

### CROSS-REFERENCE TO RELATED APPLICATIONS

This application claims the benefit of International application number PCT/EP2019/053471, filed Feb. 12, 2019 and French application number 1851306, filed Feb. 15, 2018 the entire contents of which are incorporated herein by reference.

### TECHNICAL FIELD

The invention relates to a method for preparing an irradiated platelet lysate as well as an irradiated platelet lysate obtained by such a method and a method for culturing cells using such an irradiated platelet lysate.

### BACKGROUND

The invention applies to the field of blood platelet derived products, and in particular to the field of cell culture for cultivating cells for therapeutic use, more particularly mesenchymal stem cells.

To cultivate animal cells in vitro, conventionally basal mediums of the RPMI (Roswell Park Memorial Institute), MEM (Modified Eagle Medium) or DMEM (Dulbecco Modified Eagle Medium) type are used comprising substantially mineral salts, glucose, amino acids, vitamins and nitrogenous bases. These basal mediums are generally extemporaneously supplemented with antibiotics in order to prevent bacterial contamination, L-glutamine—an unstable amino acid —, and between 1.5 and 10% foetal calf serum as a nutritive supplement.

In order to use more effective products and to avoid the use of xenogeneic products in cell cultures for therapeutic use, it has been proposed to replace the foetal calf serum with human platelet lysate. The latter has in particular the advantage of comprising a substantial quantity of growth factors such as for example TGF-beta1 (Transforming Growth Factor-beta1), EGF (Epidermal Growth Factor), PDGF-AB (Platelet-Derived Growth Factor-AB), PDGF-BB (Platelet-Derived Growth Factor-BB), IGF-1 (Insulin-like Growth Factor-1), VEGF (Vascular Endothelial Growth Factor), and FGF-2 (Fibroblast Growth Factor 2), also called bFGF (Basic Fibroblast Growth Factor).

Thus, the following human or non-human animal cells were cultivated in the presence of a platelet lysate: keratinocytes, renal epithelial cells, leukemic cell lines or solid tumour derived leukemic cell lines, as well as human primary cells such as adipocytes, stem cells of the amniotic fluid, bone marrow stem cells, chondrocytes, corneal cells, endothelial cells, keratinocytes, mesenchymal stem cells, monocytes and osteoblasts (Pons, Miriam, et al. “Human platelet lysate as validated replacement for animal serum to assess chemosensitivity.” ALTEX-Alternatives to animal experimentation (2018).

The platelet lysate is typically prepared from a suspension of platelets in plasma or in a mixture of plasma and an additive solution for platelets, that is subjected to one or more freezing/thawing cycles to release the growth factors contained in the platelets by cell lysis.

A disadvantage with such a platelet lysate is that it contains fibrinogen, coming from the plasma, in a quantity varying from about 0.5 to 3 mg/ml, in such a way that the adding of an anticoagulant to the basal medium is required in order to avoid a coagulation of the basal medium (Burnouf T, Strunk D, Koh M B, et al.: Human platelet lysate: replacing foetal bovine serum as a gold standard for human cell propagation? Biomaterials 2016; 76:371-387).

The most widely used anticoagulant is heparin. But the commercially available heparins are generally of porcine origin, in such a way that the culture medium, although devoid of foetal calf serum, still comprises a xenogeneic product.

Furthermore, at certain concentrations, heparin has a negative effect on cell proliferation (Viau, Sabrina, et al. “Pathogen reduction through additive-free short-wave UV light irradiation retains the optimal efficacy of human platelet lysate for the expansion of human bone marrow mesenchymal stem cells.” PloS one 12.8 (2017): e0181406).

Several strategies have been proposed to eliminate the coagulation power of a platelet lysate:

A first method is described in document WO 2013/003356, which consists of adding calcium chloride to the platelet lysate, inducing the conversion of the fibrinogen into fibrin and the formation of a clot. The clot is then eliminated by centrifugation in order to obtain a lysate comprising less than 0.05 mg/ml of fibrinogen. This method however generates a loss in growth factors that might be trapped in the clot.

An indirect method consists of eliminating the plasma that contains the fibrinogen from the starting platelet suspension, for example by carrying out several platelet washing cycles before preparing the platelet lysate. This strategy is for example described in document WO 2008/034803 and in document US 2012/0156306. The elimination of the plasma however generates in fact the elimination of other plasma proteins such as albumin, required for cell proliferation.

In document WO 2017/162830, it is proposed to eliminate the plasma from the starting platelet suspensions then to heat the platelet lysate between 55 and 65° C. for 20 to 40 minutes, in such a way as to obtain a quantity of fibrinogen less than 0.3 mg/ml. However, this method also reduces the quantity of the other proteins present in the platelet lysate by at least 70%, which is not desirable for a use in cell culture.

Laner-Plamberger et al. propose an original method consisting of adding the non-defibrinogenated platelet lysate to the basal medium in order to form a fibrin gel, then to destroy and eliminate this gel by vigorous agitation and centrifugation. The basal medium supplemented with platelet lysate thus obtained is devoid of fibrinogen, but still contains the other plasma proteins (J Transl Med (2015) 13:354). This method is however complex to set up in the context of a standardised and quality-controlled industrial production of platelet lysates.

In document WO 2016/193591, the platelet lysate is subjected to an irradiation with ionising radiation in order to be sterilised. According to the dose of irradiation used, the irradiated platelet lysate can comprise less than 0.4 mg/ml of fibrinogen. According to the example described, a platelet lysate was prepared from platelet concentrates comprising platelets suspended in 30% plasma and 70% preservation solution, then irradiated at a dose of 35 or 45 kGy. When the irradiated platelet lysate is added in a quantity of 8% to the basal medium, the basal medium does not coagulate.

Furthermore, document WO 2013/042095 and the article by S. Castiglia (Castiglia, Sara, et al. “Inactivated human platelet lysate with psoralen: a new perspective for mesenchymal stromal cell production in Good Manufacturing Practice conditions.” Cytotherapy 16.6 (2014): 750-763) disclose a platelet lysate obtained from buffy coat derived platelet concentrates that have been subjected to a viral inactivation by an ultraviolet radiation of the UVA type in the presence of psoralen, a DNA intercalating chemical agent. This technique of viral inactivation however has the disadvantage of having to use a chemical agent that must then be eliminated from the treated product.

Another method of obtaining a platelet lysate devoid/free of pathogens is described in the article by S. Viau (Viau, Sabrina, et al. “Pathogen reduction through additive-free short-wave UV light irradiation retains the optimal efficacy of human platelet lysate for the expansion of human bone marrow mesenchymal stem cells.” PloS one 12.8 (2017): e0181406). In this article, a platelet lysate is obtained from platelet concentrates which were subjected to/have undergone a viral inactivation by UVC radiation, in the absence of chemical agent.

In these latter documents, the platelet lysate prepared from virus-inactivated platelet concentrates/remain coagulable.

The invention proposes a new method for preparing a platelet lysate with a reduced coagulation power in the presence of calcium, while still retaining the plasma proteins required, in particular for cell proliferation.

Thus, according to a first aspect, the invention proposes a method for preparing an irradiated platelet lysate comprising the following steps:
providing a platelet lysate in order to obtain a starting platelet lysate, the starting platelet lysate comprising on the one hand platelet factors including growth factors and on the other hand plasma proteins including coagulation factors and proteins other than the coagulation factors, and
irradiating the starting platelet lysate, with UVC radiation having a wavelength comprised between 200 and 280 nm in order to obtain a platelet lysate irradiated with UVC radiation, the irradiation being arranged to retain at least 75% of the total protein concentration of the starting platelet lysate while reducing, by at least 20% the concentration of at least one of the coagulation factors including fibrinogen, factor II, factor VII, factor IX, factor X and factor XI of the starting platelet lysate.

According to a second aspect, the invention relates to the irradiated platelet lysate obtained by the method according to the first aspect of the invention.

According to a third aspect, the invention proposes a method for the culture of cells, particularly animal cells and more particularly mesenchymal stem cells, comprising the putting of the cells into contact with a nutritive composition comprising a basal medium and an irradiated platelet lysate according to the second aspect of the invention.

### BRIEF DESCRIPTION OF THE DRAWINGS

Other objects and advantages shall appear in the following description.

FIGS. 1 to 6 show, respectively, the concentrations in IGF-1, TGF-beta1, bFGF, PDGF-AB, EGF and VEGF growth factors in three batches of platelet lysate irradiated with UVC radiation, according to the dose of irradiation.

FIGS. 7 to 11 show, respectively, the concentrations of factor II, factor VII, factor IX, factor X and factor XI coagulation factors, expressed as a percentage with respect to a standard normal human plasma, in three batches of platelet lysate irradiated with UVC radiation, according to the dose of irradiation.

FIG. 12 shows the amplification factors on the 7th day of culture of mesenchymal stem cells cultivated in the presence of platelet lysate irradiated with UVC radiation according to the dose of irradiation.

FIGS. 13 to 16 show, respectively, the concentrations of bFGF, PDGF-AB, PDGF-BB and TGF-beta1 growth factor, of a non-irradiated platelet lysate (PL) and of a platelet lysate irradiated with UVC radiation at a dose of 1 J/cm² (PL-UVC).

FIGS. 17 and 18 show, respectively, the total protein concentrations and the concentrations of vitamin B12, of a non-irradiated platelet lysate (PL) and of a platelet lysate irradiated with UVC radiation at a dose of 1 J/cm² (PL-UVC).

FIG. 19 shows the amplification factors on the 7th day of culture of mesenchymal stem cells cultivated in the presence of a non-irradiated platelet lysate (PL) and of a platelet lysate irradiated with UVC radiation at a dose of 1 J/cm2 (PL-UVC).

### DETAILED DESCRIPTION

The invention relates to a method for preparing an irradiated platelet lysate for the purpose in particular of obtaining a platelet lysate having a reduced coagulation power.

“Platelet lysate” means the product of the lysis of platelets, i.e. the product obtained after the disintegration of the cell membrane of the platelets which leads to the release of molecules (growth factors, cytokines) normally contained inside platelets.

The lysis of the platelets is for example carried out by one or more freezing/thawing cycles, by the use of ultrasound or by a solvent/detergent treatment.

The method according to the invention comprises firstly a step consisting of providing a platelet lysate in order to obtain a starting platelet lysate, the starting platelet lysate comprising on the one hand platelet factors including growth factors and on the other hand plasma proteins including coagulation factors and proteins other than the coagulation factors.

The platelet lysate is produced from platelets in suspension in a liquid comprising plasma. Such a suspension of platelets is for example a platelet concentrate or a mixture of platelet concentrates, a buffy coat layer, or a mixture of buffy coat layers, a platelet-rich plasma or a mixture of platelet-rich plasmas.

More particularly, the platelet suspension is a platelet concentrate derived from apheresis or prepared from a blood donation or a mixture of platelet concentrates derived from apheresis or prepared from blood donations.

For example, the mixture comprises between 4 and 50 platelet concentrates, in particular between 5 and 30 platelet concentrates.

The preparation of a platelet lysate from a mixture of several platelet concentrates, in particular more than four platelet concentrates, is advantageous because it makes it possible to standardise the platelet lysate, i.e. to homogenise the concentration of the various components thereof, in particular the concentration of growth factors (Viau S, Eap S, et al. A standardized and characterized clinical grade human platelet lysate for efficient expansion of human bone marrow mesenchymal stem cells. Cytotherapy. May 2017, Volume 19, Issue 5, Supplement, Page S195).

Indeed, the concentrations of growth factors of a platelet lysate depend on the initial platelet donor.

The platelet concentrates are either fresh, i.e. qualified to be transfused to a patient, or expired, i.e. stored for 5 days or more after the preparation thereof and no longer suitable to be transfused to a patient.

Such platelet concentrates comprise platelets in suspension in a liquid medium containing plasma.

For example, the liquid medium comprises only plasma. According to another example, the liquid medium further comprises a preservation solution of the platelets, such as the solution SSP+(Maco Pharma) or Intersol® (Fresenius Kabi).

In a particular example, the liquid medium comprises from 20% to 100%, in particular 30% plasma and from 0% to 80%, in particular 70% platelet preservation solution.

The lysis of a suspension of platelets comprising platelets in plasma provides a starting platelet lysate comprising on the one hand platelet factors normally contained inside platelets and on the other hand constituents of the plasma.

The plasma comprises water at 90%, salts such as sodium, chlorine and calcium, lipids such as triglycerides and cholesterol, hormones, vitamins such as vitamin B12 and vitamin D and proteins such as albumin, immunoglobulins, the coagulation factors such as fibrinogen, antithrombin III involved in the coagulation chain, globulins, interleukins and interferons.

Thus, the starting platelet lysate to which the method of the invention is applied comprises in particular on the one hand platelet factors including growth factors and on the other hand plasma proteins including coagulation factors and proteins other than the coagulation factors.

These growth factors are in particular TGF-beta1, EGF, PDGF-AB, IGF-1, VEGF and bFGF. Other growth factors that can be found in the platelet lysate are in particular CTGF (Connective Tissue Growth Factor) and SDF-1 alpha (Stromal Cell-Derived Factor-1 alpha). These growth factors are referred as endogenous.

“Endogenous substance” means any substance produced by the platelets or comprised in the starting platelet suspension used to prepare the platelet lysate, in opposition to an exogenous substance introduced into the platelet lysate or into the starting platelet suspension.

For example, a platelet lysate produced from lysis by freezing/thawing of a platelet suspension comprises the following concentrations of growth factor:

TABLE 1

| | | |
|---|---|---|
| bFGF | 110-180 | pg/ml |
| PDGF-AB | 20,000-45,000 | pg/ml |
| PDGF-BB | 12,000-15,000 | pg/ml |
| IGF-1 | 25-150 | pg/ml |
| VEGF | 500-1,000 | pg/ml |
| EGF | 1,600-3,000 | pg/ml |
| TGFbeta1 | 30,000-80,000 | pg/ml |

The starting platelet lysate comprises on the other hand plasma proteins including coagulation factors and proteins other than the coagulation factors.

The coagulation factors are in particular fibrinogen, factor II, factor VII, factor IX, factor X and factor XI. Other coagulation factors are factor V and factor VIII.

The other proteins of the plasma other than the coagulation factors are in particular albumin and antithrombin III, a protein involved in the coagulation chain.

The total quantity of proteins of the starting platelet lysate therefore depends on the percentage of plasma in the starting platelet suspension, before lysis of the platelets.

For example, a starting platelet lysate produced from lysis by freezing/thawing of platelet concentrates comprising platelets suspended in 100% plasma includes in particular the following components:

TABLE 2

| | | |
|---|---|---|
| Total proteins (mg/ml) | 60-80 | mg/ml |
| Fibrinogen (mg/ml) | 0.5-1.5 | mg/ml |
| Vitamin B12 | 250-300 | pg/ml |

In another example, a starting platelet lysate produced from lysis by freezing/thawing of platelet concentrates comprising platelets suspended in 30% plasma and 70% of a preservation solution of the platelets comprises in particular the following components:

TABLE 3

| | | |
|---|---|---|
| Total proteins (mg/ml) | 20-30 | mg/ml |
| Fibrinogen (mg/ml) | 0.45-0.5 | mg/ml |
| Vitamin B12 | 150-170 | pg/ml |

The step of providing a platelet lysate is to be understood as making a platelet lysate available. That is to say the method of the invention is implemented on a platelet lysate produced beforehand by lysis of the platelets of a platelet suspension.

After the step of providing a starting platelet lysate, the method according to the invention comprises the step of irradiating the starting platelet lysate, with UVC radiation having a wavelength comprised between 200 and 280 nm in order to obtain a platelet lysate irradiated with UVC radiation, the irradiation being arranged to retain at least 75% of the total protein concentration of the starting platelet lysate while reducing, by at least 20% the concentration of at least one of the coagulation factors including fibrinogen, factor II, factor VII, factor IX, factor X and factor XI of the starting platelet lysate.

“UVC radiation” refers to a non-ionising electromagnetic radiation, i.e. a radiation that is not able to cause the ionisation of atoms or of molecules. UVC radiation has a wavelength comprised between 200 and 280 nm, in particular 254 nm.

By retaining more than 75% of the total proteins of the starting platelet lysate, the platelet lysate thus irradiated with UVC radiation can be used as a supplement for basal medium for cell culture. In particular, the albumin which represents more than 50% of the proteins in the plasma and which is a particularly important nutrient in cell culture, is retained at at least 80% with respect to the starting platelet lysate.

In particular, at least 80%, and more particularly at least 90% of the total protein concentration in the platelet lysate irradiated with UVC radiation is retained with respect to the starting platelet lysate.

The platelet lysate irradiated with UVC radiation comprises a total protein concentration ranging from 20 to 80 mg/ml, according to the concentration of plasma of the starting platelet suspension.

For example, a platelet lysate produced from lysis by freezing/thawing of platelet concentrates comprising platelets suspended in 100% plasma then irradiated with UVC radiation comprises a total protein concentration ranging from about 55 mg/ml to about 80 mg/ml.

According to another example, a platelet lysate produced from lysis by freezing/thawing of platelet concentrates comprising platelets suspended in 30% plasma then irradiated with UVC radiation comprises a total protein concentration ranging from about 18 mg/ml to about 30 mg/ml.

In addition, by reducing by at least 20% the concentration of one of the coagulation factors including fibrinogen, factor II, factor VII, factor IX, factor X and factor XI, the coagulation power of the platelet lysate irradiated with UVC radiation is reduced.

That is to say a basal medium comprising calcium, for example about 0.2 g/l of calcium chloride will not coagulate in the presence of platelet lysate irradiated with UVC radiation or coagulate from a concentration of platelet lysate irradiated with UVC radiation in the basal medium higher than a starting platelet lysate from which the basal medium coagulates.

For example, the basal medium alphaMEM coagulates in the presence of 5% or more of starting platelet lysate, while this medium coagulates from 10% of platelet lysate irradiated with UVC radiation.

In particular, the platelet lysate irradiated with UVC radiation is added in a range from 2 to 25%, in particular in the range from 5 to 15%, and more particularly in the range from 8 to 10% in a basal medium.

The platelet lysate irradiated with UVC radiation having a reduced coagulation power, the use thereof as a basal medium supplement for cell culture is possible, in certain concentrations, without using any anticoagulant such as heparin.

In particular, the irradiating with UVC radiation is arranged to reduce, by at least 20% the concentration of each one of the coagulation factors including fibrinogen, factor II, factor VII, factor IX, factor X and factor XI of the starting platelet lysate.

For example, a platelet lysate produced from lysis by freezing/thawing of platelet concentrates comprising platelets suspended in 30% plasma then irradiated with UVC radiation comprises the following plasma constituents.

TABLE 4

| | | |
|---|---|---|
| Total proteins (mg/ml) | 14-30 | mg/ml |
| Fibrinogen (mg/ml) | <0.4 | mg/ml |
| Vitamin B12 | 125-140 | pg/ml |

In addition, the starting platelet lysate comprises in particular the TGF-beta1, EGF, PDGF-AB, IGF-1, VEGF and bFGF endogenous growth factors.

The irradiation with UVC radiation is in particular arranged to retain at least 80% of the concentration of one of the growth factors including IGF-1, PDGF-AB, EGF and VEGF of the starting platelet lysate, in particular in order to be able to use the platelet lysate irradiated with UVC radiation as a basal medium supplement.

In particular, the irradiation with UVC radiation is arranged to retain at least 80% of the concentration of each one of the growth factors including IGF-1, PDGF-AB, EGF and VEGF, in particular in order to be able to use the platelet lysate irradiated with UVC radiation as a basal medium supplement.

For example, a platelet lysate produced from lysis by freezing/thawing of platelet concentrates comprising platelets suspended in 30% plasma then irradiated with UVC radiation comprises the following growth factors.

TABLE 5

| | | |
|---|---|---|
| bFGF | 88-140 | pg/ml |
| PDGF-AB | 16,000-45,000 | pg/ml |
| PDGF-BB | 6,400-12,000 | pg/ml |
| IGF-1 | 20-120 | pg/ml |
| VEGF | 400-900 | pg/ml |
| EGF | 1,300-2,800 | pg/ml |
| TGF-beta1 | 24,000-70,000 | pg/ml |

The platelet lysate irradiated with UVC radiation thus retains its interest for a use in cell culture or other application for which the growth factors have an interest.

The irradiation with UVC radiation is in particular arranged to retain at least 75%, particularly at least 80%, of the amplification factor of the mesenchymal stem cells cultivated for 7 days in a basal medium supplemented with starting platelet lysate.

According to a particular embodiment, the irradiation with UVC radiation is carried out at a dose comprised between 0.01 to 2 J/cm², particularly between 0.5 J/cm² and 1.5 J/cm², and more particularly at 1 J/cm².

A dose of UVC radiation less than 0.01 J/cm² is not sufficient to degrade the coagulation factors present in the starting platelet lysate. A dose of UVC radiation greater than 2 J/cm² damages the growth factors generating a substantial loss in cell proliferation.

In particular, the starting platelet lysate is irradiated with UVC radiation in the liquid state.

For example, the starting platelet lysate in the liquid state is contained in a UVC-permeable recipient, such as a UVC-permeable irradiation bag. A UVC-permeable irradiation bag is in particular made from a material that does not have an adsorption maximum in the range from 200 to 280 nm. The irradiation bag is in particular made from vinyl acetate or from polytetrafluoroethylene.

The irradiation bag containing the starting platelet lysate is then disposed in an UVC illumination device. The bag is stirred orbitally during the irradiation with UVC radiation, so as to homogeneously irradiate all of the platelet lysate.

Alternatively, the irradiating with UVC radiation of the starting platelet lysate is carried out under flow condition.

According to a particular embodiment, the method comprises, prior to the irradiation with UVC radiation, a step of filtering the starting platelet lysate through a filter with a porosity of 0.65 μm or less, particularly 0.45 μm or less.

This step of filtering makes it possible to eliminate any cellular debris coming from the step of lysing of the platelets and which could hinder the irradiation of the platelet lysate.

According to another particular embodiment, the method comprises, after the irradiation with UVC radiation, a step of sterilising filtration of the platelet lysate irradiated with UVC radiation through a filter with a porosity of 0.45 μm or less, particularly 0.22 μm or less.

This step of filtering through a sterilising filter makes it possible to retain bacteria having a size greater than 0.22 μm and, advantageously combined with the irradiation with UVC radiation, makes it possible to obtain a platelet lysate having a reduced risk of bacterial and viral contamination.

In order to further reduce the coagulation power of the platelet lysate irradiated with UVC radiation, the method according to the invention advantageously comprises a step of irradiating the platelet lysate with ionising radiation having a wavelength less than or equal to 100 nm, particularly less than 10 nm. Ionising radiation having a wavelength less than or equal to 100 nm comprises X-UV rays having a wavelength ranging from 10 nm to 100 nm, X rays having a wavelength ranging from 10 μm to 10 nm and gamma rays having a wavelength less than 10 μm.

A method of irradiating a platelet lysate with ionising radiation is in particular described in patent application WO 2016/193591.

In such a method, prior to the step of irradiation by ionising radiation, the method comprises a step of freezing the platelet lysate in order to irradiate with ionising radiation the platelet lysate in the frozen state.

In particular, the freezing of the platelet lysate is carried out at a temperature comprised between −10° C. and −196° C., in particular about −20° C. or about −80° C.

Alternatively, the platelet lysate is irradiated by ionising radiation in a freeze-dried state.

For the irradiation with ionising radiation in a frozen state, the platelet lysate is contained in a recipient that resists freezing and in particular in a bag that resists freezing. The material that is resistant to freezing is in particular ethylene vinyl acetate, polyethylene or a fluoropolymer such as fluorinated ethylene-propylene.

According to a particularly advantageous embodiment, the irradiation with ionising radiation is carried out after the irradiation with UVC radiation, i.e. the irradiation with ionising radiation is carried out on the platelet lysate irradiated with UVC radiation.

More advantageously, the irradiation with ionising radiation is carried out after the irradiation with UVC radiation and after the sterilising filtration of the platelet lysate irradiated with UVC radiation, i.e. on the platelet lysate irradiated with UVC radiation then filtered in a sterile manner.

In this case, the step of irradiating with ionising radiation is carried out on the platelet lysate in its final conditioning, in particular in a storage bag. The storage bag is for example made from a material that resists freezing and the irradiation with ionising radiation such as ethylene vinyl acetate.

Alternatively, the irradiation with ionising radiation is carried out prior to the irradiation with UVC radiation on the starting platelet lysate.

According to an embodiment, the ionising radiation is a gamma radiation having a wavelength less than or equal to 10 μm.

Gamma radiation is an electromagnetic radiation comprised of high-energy photons, of about 1.6 MeV. It is for example emitted by a cobalt 60 source.

According to another aspect, the invention relates to an irradiated platelet lysate obtained by the method according to the first aspect of the invention.

The platelet lysate prepared according to the method of preparing of the invention has a particular growth factor and protein profile.

In particular, the irradiation with UVC radiation impacts certain growth factors that are not impacted or less impacted by an irradiation with ionising radiation, for example. These growth factors include in particular the EGF, TGF-beta1 and PDGF-BB factors.

For example, the platelet lysate irradiated with UVC radiation comprises a concentration of endogenous EGF growth factor less than 2,800 pg/ml, and/or a concentration of endogenous TGF-beta1 growth factor less than 70,000 pg/ml, in particular less than 40,000 ng/ml, and/or a concentration of endogenous PDGF-BB growth factor less than 12,000 pg/ml.

The same applies for vitamin B12 impacted by the UVC radiation but not by the ionising radiation.

For example, the platelet lysate irradiated with UVC radiation comprises a concentration of vitamin B12 reduced by 10 to 30% with respect to the starting platelet lysate. In particular, the concentration of vitamin B12 is comprised in the range from 125 to 140 pg/ml.

Certain growth factors or proteins are not or are hardly impacted by the irradiation with UVC radiation or by the irradiation with ionising radiation.

Thus, the platelet lysate irradiated with UVC radiation comprises a concentration of PDGF-AB growth factor comprised in the range from 16,000 to 45,000 pg/ml.

Certain growth factors or proteins are not impacted by the irradiation with UVC radiation, but are impacted by the irradiation with ionising radiation.

For example, antithrombin III, a protein involved in the coagulation chain is only slightly impacted by the irradiation with UVC radiation.

Certain growth factors are impacted by the irradiation with UVC radiation as by the irradiation with ionising radiation.

Thus, the platelet lysate irradiated with UVC radiation comprises a concentration of bFGF endogenous growth factor less than 140 pg/ml.

The platelet lysate irradiated with UVC radiation further comprises a concentration of fibrinogen less than 0.4 mg/ml.

The irradiation with UVC radiation does not have any notable impact on the total protein concentration of the platelet lysate.

Thus, the platelet lysate irradiated with UVC radiation comprises a total protein concentration comprised between 14 and 80 mg/ml, according to the initial quantity of plasma.

More particularly, the total protein concentration in a platelet lysate produced from lysis by freezing/thawing of platelet concentrates comprising platelets suspended in 100% plasma, irradiated with UVC radiation comprises a total protein concentration ranging from about 55 mg/ml to about 80 mg/ml.

The total protein concentration in a platelet lysate produced from lysis by freezing/thawing of platelet concentrates comprising platelets suspended in 30% plasma, irradiated with UVC radiation comprises a total protein concentration ranging from about 18 mg/ml to about 30 mg/ml.

According to a third aspect, the invention relates to a method for the culture of cells, particularly of animal cells, and more particularly mesenchymal stem cells, comprising the putting of the cells into contact with a nutritive composition comprising a basal medium and an irradiated platelet lysate according to the second aspect of the invention.

The method applies for example to the culture of human or non-human animal cells, such as keratinocytes, epithelial cells, leukemic cell lines or solid tumour derived leukemic cell lines, adipocytes, stem cells of the amniotic fluid, bone marrow stem cells, chondrocytes, corneal cells, endothelial cells, mesenchymal stem cells, monocytes, osteoblasts and natural killer cells.

The mesenchymal stem cells are for example human mesenchymal stem cells derived from bone marrow or umbilical cord blood.

According to a particular embodiment, the nutritive composition comprises from 2% to 25%, in particular from 5% to 15%, and more particularly from 8 to 10% of irradiated platelet lysate according to the invention.

In particular, the irradiated platelet lysate is added extemporaneously in a preliminary manner to the basal medium so as to form the nutritive composition.

As the irradiated platelet lysate has a reduced coagulation power, it is not necessary to add to the nutritive composition an anticoagulant of the heparin type in order to prevent the coagulation thereof and maintain it in a liquid state.

Thus, according to an embodiment of the method for the culture of cells, particularly animal cells, the nutritive composition is in liquid form and is free of anticoagulant.

### Example 1: Production in the Laboratory of Platelet Lysate Irradiated with UVC Radiation

1.1. Preparation of a Platelet Lysate

A batch of platelet lysate is prepared as described hereinbelow.

Platelet concentrates (20 platelet concentrates) comprising 70% Intersol® preservation solution and 30% plasma were prepared from a mixture of five buffy coats and retained in storage bags.

The storage bags were frozen at −80° C. for a duration of about 24 hours before being thawed at 4° C. for about 24 hours.

The thawed storage bags are then centrifuged at a speed of 5,000 g for 10 minutes so as to separate the supernatant comprising the platelet lysate from the sediment comprising the cellular debris.

The supernatant of each one of the storage bags is transferred into a mixture bag so as to as to obtain a mixture of platelet lysates (PL).

1.2. Irradiation with UVC

The mixture of platelet lysates is transferred, by volume of 500 ml, into irradiation bags. The air and all the bubbles are eliminated from the irradiation bags.

The irradiation bags are then irradiated with an UVC illumination device (Macotronic UV, Maco Pharma, France), at different doses (0-3.2 J/cm²). The irradiation bags are stirred at a speed of 110 rpm.

After irradiation with UVC radiation, the contents of the irradiation bags are re-mixed in a transfer bag.

1.3. Dosages of Cytokines

The following tests, carried out on three batches, were carried out in order to characterise the platelet lysates irradiated at different UVC doses:
Dosages using ELISA kits of IGF-1 (ref. DG100/batch 341313) and of TGF-Beta1 (ref. DB100B/batch 340010),
Dosages using ELISA kits of bFGF (ref. DFB50/batch P104841), of PDGF-AB (ref. DHD00C/batch P101565), of EGF (ref. DEG00/batch 339998), and of VEGF (ref. DVE00/batch P100719)

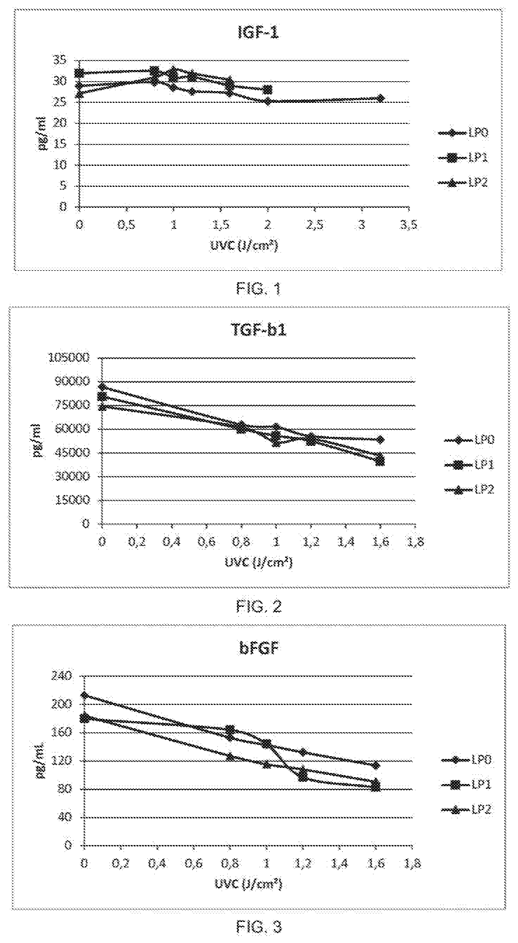

The results, shown in FIG. 1, show that the concentration of IGF-1 in the platelet lysate is not impacted by the irradiation with UVC.

The average of the three batches makes it possible to conclude that the concentration of PDGF-AB is little or not affected by the irradiation with UVC (FIG. 4).

On the other hand, FIGS. 2, 3, 5 and 6 show that the concentration of TGF-beta1, bFGF, EGF and VEGF, and decreases as the dose of UVC increases and this, from 0.8 J/cm².

The losses in EGF and VEGF are not as substantial compared to the losses in TGF-beta1 and bFGF. A loss of 23% and 24% respectively is observed for TGF-beta1 and bFGF at 0.8 J/cm² (FIGS. 2 and 3), and up to 50% and 44% respectively at 1.6 J/cm² (FIGS. 5 and 6).

1.4. Dosages of Plasma Factors

Biochemical dosages, carried out on the three same batches, were carried out in order to characterise the platelet lysates irradiated at different UVC doses.

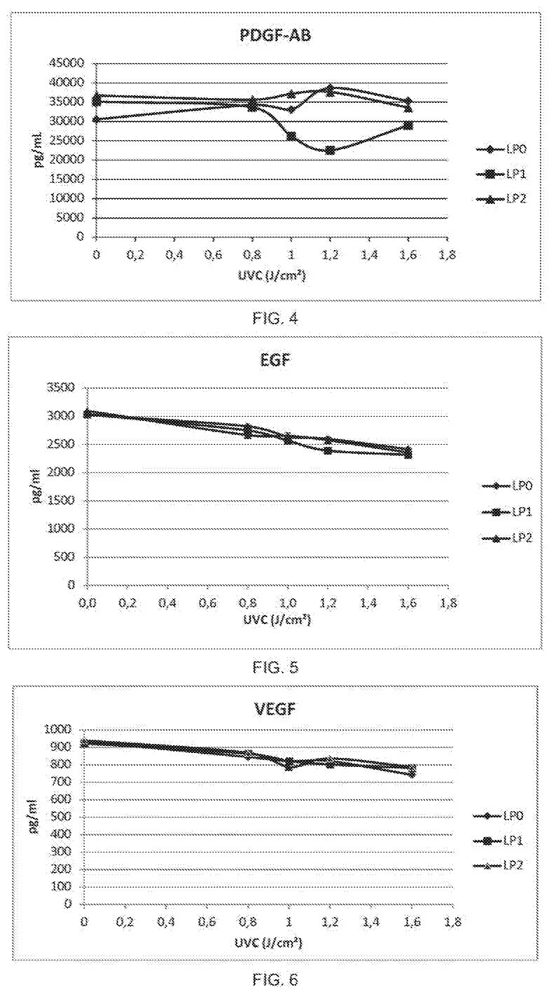

The results of the dosages of the plasma factors in the platelet lysate after irradiation with UVC, shown in FIGS. 7 to 11, show that the UVC have an effect on factors II, VII, IX, X, and XI. As for TGF-beta1, the concentration of these plasma factors decreases according to the UVC dose delivered during the irradiation.

1.5. Impact of the Irradiation with UVC Radiation on the Effectiveness of the PL (Proliferation of MSCs)

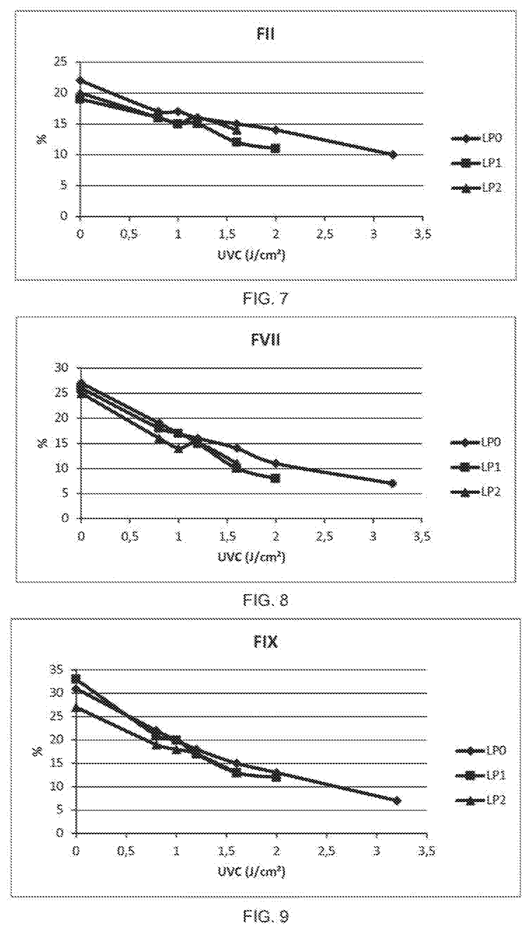

Proliferation tests, carried out on three batches, were carried out in order to characterise the platelet lysates irradiated at the different doses (FIG. 12).

The mesenchymal stem cells (MSCs) were seeded in 6-well plates (triplicates for each condition) with 2,500 cells/cm².

All the platelet lysates were used at 8% in an alphaMEM medium. The experiment was carried out twice.

FIG. 12 shows that the two experiments generate the same proliferation profile of the MSCs in contact with the platelet lysate irradiated with UVC radiation.

On the one hand, it can be noted that the controls of non-irradiated platelet lysate are very similar. In addition, a plateau is observed from 0 J/cm² to 1.2 J/cm² where the proliferation of the MSCs does not seem to be impacted. Between 1.2 J/cm² and 1.6 J/cm² the proliferation of the MSCs starts to visibly decrease.

### Example 2: Industrial Production of Platelet Lysate Irradiated with UVC Radiation

2.1 Preparation of the Platelet Lysate

Several batches of platelet lysate (PL) were produced as described in example 1.1 hereinabove, from platelet concentrates comprising platelets suspended in 30% plasma and 70% of an additive solution.

2.2 Irradiation with UVC Radiation

The mixture of platelet lysates is filtered through a filter with a porosity of 0.45 μm before being irradiated with UVC radiation.

The mixture of filtered platelet lysates is transferred, by volume of 500 ml, into irradiation bags. The air and all the bubbles are eliminated from the irradiation bags.

The irradiation bags are then irradiated with a UVC illumination device (Macotronic UV, Maco Pharma), at a dose of 1 J/cm². The irradiation bags are stirred at a speed of 110 rpm.

After irradiation with UVC radiation, the contents of the irradiation bags are re-mixed in a transfer bag and the mixture of the platelet lysates irradiated with UVC radiation is filtered through a sterilising filter with a porosity of 0.2 μm in order to form a batch of platelet lysate irradiated with UVC (PL-UVC).

2.3 Dosage of Cytokines

In samples of PL and PL-UVC, the quantity of bFGF (ref.SFB50/lot P116487), PDGF-AB (ref.SHD00C/lot P122623), PDGF-BB (ref.SBB00/lot P116857) and TGF-beta1 (ref.SB100B/lot P119433) is dosed, using commercial Elisa kits.

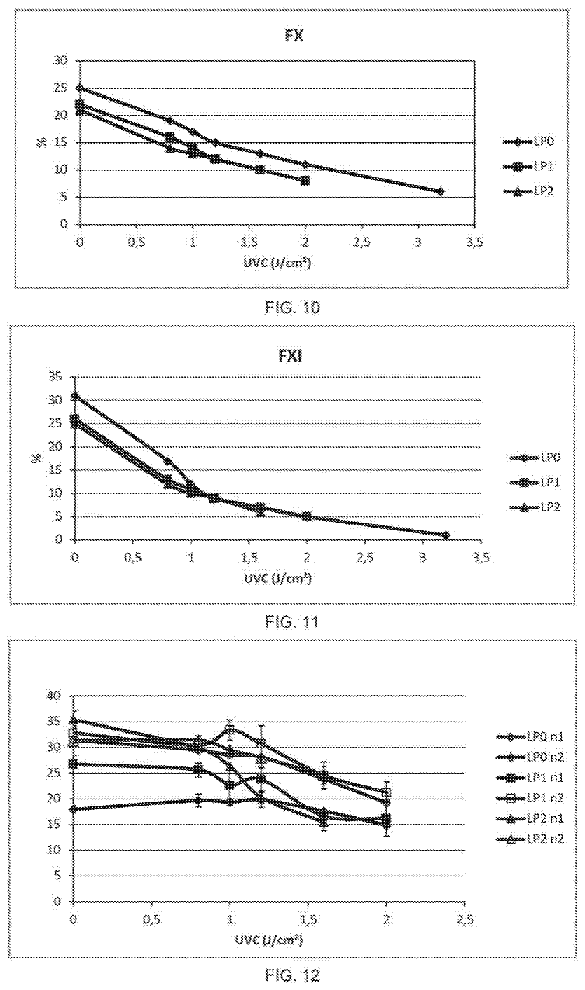

In FIG. 13, the concentration of bFGF is impacted by the irradiation with UVC radiation. Indeed, an average loss of 23% of concentration of bFGF after irradiation with UVC is observed.

In FIG. 14, the concentration of PDGF-AB is slightly impacted by the irradiation with UVC radiation. A loss of 6% of the concentration of PDGF-AB is observed caused by the effect of the irradiation with UVC.

In FIG. 15, the concentration of PDGF-BB is strongly impacted by the irradiation with UVC radiation. Indeed, an average loss of 24% of the concentration of PDGF-BB after irradiation with UVC is observed.

In FIG. 16, the concentration of TGF-beta1 is impacted by the irradiation with UVC radiation. Indeed, an average loss of 35% of concentration of TGF-beta1 is observed, caused by the irradiation with UVC radiation.

According to these results, only the concentration of PDGF-AB is slightly impacted by the irradiation with UVC radiation (6% loss), on the other hand the concentrations of bFGF and TGF-beta1 decrease by about 30%, and the concentration in PDGF-BB decreases by about 20% after treatment with UVC for the dose studied.

2.4 Dosage of Proteins

The dosage of proteins is carried out using a BCA kit (UP40840/Q05KL03).

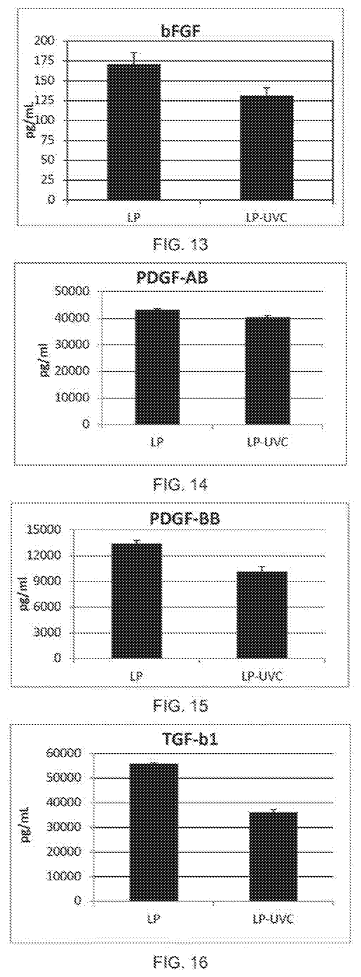

No notable effect of the irradiation with UVC radiation is observed on the concentration of proteins (FIG. 17).

2.5 Biochemical Dosages and Coagulation Factors

The biochemical analyses were carried out on the following 12 elements:

Biochemical Dosages
Total proteins
Serum albumin
Calcium
Sodium
Fibrinogen
D-dimers
Mycoplasms
Vitamin B12
Vitamin D
Chlorine
Iron
Cholesterol

Among the 12 elements dosed, only vitamin B12 is impacted by the irradiation with UVC radiation. Indeed, an average loss of 18.5% of the concentration of vitamin B12 is observed caused by the irradiation with UVC radiation (FIG. 18).

The concentration of coagulation factors (Factors II, VII, IX, X, XI) and of antithrombin III, protein involved in coagulation, is impacted by the irradiation with UVC radiation.

It is however observed that the antithrombin III is only slightly impacted by the irradiation with UVC radiation (average loss of 5% with respect to the non-irradiated platelet lysate) in comparison with other coagulation factors (Table 6 hereinbelow).

TABLE 6
Percentages of difference in concentration
of proteins with respect to the starting PL

| Proteins | PL-UVC |
|---|---|
| Factor II (FII) | −24.6% |
| Factor VII (FVII) | −10.6% |
| Factor IX (FIX) | −52.4% |
| Factor (FX) | −40.3% |
| Factor XI (FXI) | −66.7% |
| antithrombin III (ATIII) | −6.1% |

2.6 Gelation Test

The test was carried out at different concentrations of platelet lysate in the basal medium alphaMEM without heparin: 2.5%, 5%, 8%, 10%, 15%, and 20%.

TABLE 7

| % PL | 2.5% | 5% | 8% | 10% | 15% | 20% |
|---|---|---|---|---|---|---|
| PL | − | + | ++ | +++ | +++ | +++ |
| PL-UVC | − | − | − | + | +++ | +++ |

−: no gelation
+: gelling effect but quasi liquid
++: gelled to 50%
+++: gelled to 100%

For the non-irradiated platelet lysate (PL), with a low percentage of platelet lysate (2.5%), there is no gelling effect. The results show that the more the percentage of platelet lysate present in the basal medium increases, the more substantial the gelation of the basal medium is.

At 5% of PL, the basal medium already has a gelled effect, and at 8% of platelet lysate, the basal medium is gelled to 50%.

The basal medium containing PL-UVC does not begin to gel until concentrations of PL-UVC of 10%.

In conclusion, it is noted that the sole irradiation with UVC radiation degrades the coagulation power of the PL.

2.7 Proliferation Tests

The cells used are bone-marrow derived primary human mesenchymal stem cells (MSC) coming from two different donors (M065 and M068). Two female experimenters carried out the blind experiment.

The first day, 6-well plates were seeded with 2,500 cells/cm² in triplicate. The hMSCs are at P3.

The basal medium used is the alphaMEM medium and the samples of platelet lysates at 8% are those of table 7.

The medium is changed every 3 days. After 7 days of cell culture, the cells are counted with the ViCell.

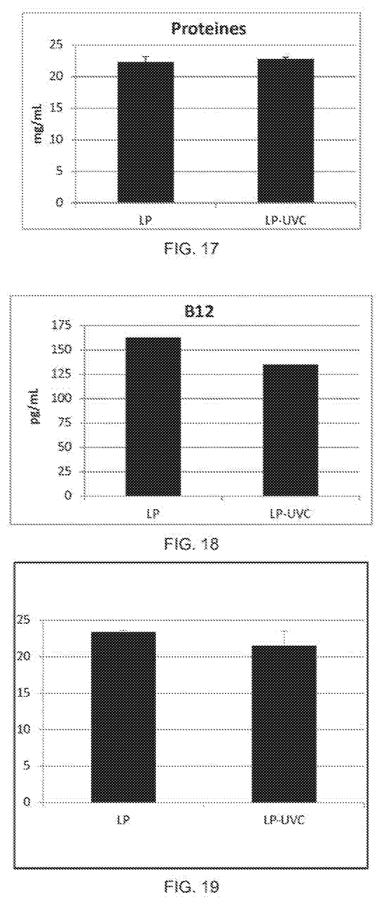

Only a small impact of the irradiation with UVC radiation is observed on the effectiveness of the platelet lysate: loss of 8% of the cell proliferation caused by the UVCs (FIG. 19).

What is claimed is:

1. A method for preparing an irradiated platelet lysate comprising the following steps:
providing a platelet lysate to obtain a starting platelet lysate, the starting platelet lysate comprising platelet factors including growth factors and further comprising plasma proteins including a plurality of coagulation factors and proteins other than the coagulation factors,
irradiating the starting platelet lysate, with UVC radiation having a wavelength comprised between 200 and 280 nm in order to obtain a platelet lysate irradiated with UVC radiation, the irradiation being arranged to retain at least 75% of the total protein concentration of the starting platelet lysate while reducing, by at least 20% the concentration of at least one of the coagulation factors including fibrinogen, factor II, factor VII, factor IX, factor X and factor XI of the starting platelet lysate.

2. The method according to claim 1, wherein the irradiation with UVC radiation is arranged to reduce, by at least 20% the concentration of each one of the coagulation factors including fibrinogen, factor II, factor VII, factor IX, factor X and factor XI in the starting platelet lysate.

3. The method according to claim 1, wherein the starting platelet lysate comprises at least TGF-beta1, EGF, PDGF-AB, IGF-1, VEGF and bFGF endogenous growth factors, and in that the irradiation with UVC radiation is arranged to retain at least 80% of the concentration of one of growth factors including IGF-1, PDGF-AB, EGF and VEGF, and at least 80% of the concentration of each one of the growth factors including IGF-1, PDGF-AB, EGF and VEGF, of the starting platelet lysate.

4. The method according to claim 1, wherein the irradiation with UVC radiation is carried out at a dose comprised between 0.01 to 2 J/cm², particularly between 0.5 J/cm² and 1.5 J/cm², and more particularly at 1 J/cm².

5. The method according to claim 1, wherein the starting platelet lysate is irradiated with UVC radiation in a liquid state.

6. The method according to claim 1, wherein prior to the irradiation with UVC radiation, a step of filtering the starting platelet lysate through a filter with a porosity of 0.65 μm or less, particularly 0.45 μm or less.

7. The method according to claim 1, wherein after the irradiation with UVC radiation, a step of sterilising filtration of the platelet lysate irradiated with UVC radiation through a filter with a porosity of 0.45 μm or less, particularly 0.22 μm or less.

8. The method according to claim 1, wherein after the irradiation with UVC radiation, a step of irradiating the platelet lysate with ionising radiation having a wavelength less than or equal to 100 nm, particularly less than 10 nm, and in particular with gamma radiation having a wavelength less than or equal to 10 pm.

9. The method according to claim 8, wherein prior to the step of irradiating with ionising radiation, a step of freezing the platelet lysate in order to irradiate with ionising radiation the platelet lysate in the frozen state.

10. The method according to claim 8, wherein the irradiation with ionising radiation is carried out at an absorbed dose comprised in the range from 20 kGy to 75 kGy, in particular from 35 kGy to 55 kGy.

11. An irradiated platelet lysate obtained by the method according to claim 1, wherein the irradiated platelet lysate comprises a total protein concentration comprised between 18 and 80 mg/ml, a concentration of fibrinogen less than 0.4 mg/ml, and a concentration in endogenous TGF-beta1 growth factor less than 70,000 pg/ml.

12. The irradiated platelet lysate according to claim 11, wherein the irradiated platelet lysate comprises a concentration of endogenous bFGF growth factor less than 140 pg/ml, a concentration of endogenous EGF growth factor less than 2,800 pg/ml, and a concentration of endogenous PDGF-BB growth factor less than 12,000 pg/ml.

13. The irradiated platelet lysate according to claim 11, wherein the irradiated platelet lysate comprises a concentration of vitamin B12 comprised between 120 and 140 pg/ml.

14. A method for the culture of animal cells, in particular mesenchymal stem cells, comprising the putting of the cells into contact with a nutritive composition comprising a basal medium and an irradiated platelet lysate according to claim 11.

15. The method according to claim 14, wherein the nutritive composition is in liquid form and is free of anticoagulant.
